(19) United States
(12) Patent Application Publication
(10) Pub. No.: US 2013/0296741 A1
(43) Pub. Date: Nov. 7, 2013

(54) ANKLE-FOOT ORTHOTIC DEVICES WITH INTEGRATED VIBROTACTILE BIOFEEDBACK AND RELATED METHODS

(71) Applicant: NORTH CAROLINA STATE UNIVERSITY, Raleigh, NC (US)

(72) Inventors: Michael Bruce Wiggin, Raleigh, NC (US); Audrey Elizabeth Westbrook, Four Oaks, NC (US); Gregory Stephen Sawicki, Durham, NC (US); Adam Kenneth Willson, Cary, NC (US); Tojan Bassam Rahhal, Raleigh, NC (US); Whitney Celeste Barnette, Jacksonville, NC (US); Kristen Elizabeth Lasater, Newport, NC (US)

(73) Assignee: NORTH CAROLINA STATE UNIVERSITY, Raleigh, NC (US)

(21) Appl. No.: 13/886,247

(22) Filed: May 2, 2013

Related U.S. Application Data
(60) Provisional application No. 61/641,562, filed on May 2, 2012.

(51) Int. Cl. A61B 5/11

(52) U.S. Cl. CPC A61B 5/112
USPC 600/595

(57) ABSTRACT

Ankle-foot orthotic devices with integrated vibrotactile biofeedback and related methods are disclosed. According to an aspect, an ankle-foot orthotic device may include one or more sensors configured to determine a gait phase of a user and to determine a movement of the user. Further, the device may include a controller configured to communicate feedback to the user based on the determined gait phase and the determined movement of the user.

### CROSS REFERENCE TO RELATED APPLICATION

This application claims the benefit of U.S. Provisional Patent Application Ser. No. 61/641,562, filed May 2, 2012; the disclosure of which is incorporated herein by reference in its entirety.

### TECHNICAL FIELD

The subject matter described herein relates to orthotic devices. More particularly, the subject matter described herein relates to ankle-foot orthotic (AFO) devices with integrated vibrotactile biofeedback.

### BACKGROUND

Ankle foot orthoses are devices that are worn by individuals to aid in walking and gait rehabilitation. For example, individuals who exhibit muscular or neuromuscular impairment about the lower limb have an increased risk of foot drop, commonly resulting in falls, tripping, or further injury. This brings about the increased need for stability and awareness during locomotion due to decreased sensation from the impairment.

Most existing AFO devices only work to restrict angular movement of the ankle joint to prevent foot drop or other abnormal conditions during walking caused by impairment. Other AFO devices contain active components, such as motors, to assist in locomotion. Using active mechanisms requires an onboard power supply, resulting in increased weight and intricacy of such devices. Accordingly, in light of these difficulties, there exists a need for improved AFO devices and related techniques.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Disclosed herein are AFO devices for reducing the risk of injury, fall, or foot drop by dynamic feedback and an integrated vibrotactile response. In accordance with an aspect, an AFO device with integrated vibrotactile biofeedback includes the following components: an advanced plastic or composite AFO with integrated monitoring and control system, kinematic sensor, one or more biosensors, and a feedback mechanism. Example kinematic sensors include, but are not limited to, a force sensor, pressure sensor, accelerometer, and the like. Example biosensors include, but are not limited to, a goniometer, flex sensor, and the like. Example feedback mechanisms include, but are not limited to, a speaker, vibrator, and the like.

In an aspect, AFO devices disclosed herein may assist in preventing foot drop by providing direct feedback through an integrated monitoring and control system. In another aspect, use of an advanced composite or plastic structure for an AFO brace, the AFO device can be set to provide assistive dorsiflexion. In yet another aspect, one or more sensors can be integrated into an AFO brace with an attached circuit to monitor the AFO device and user for alerting the user in instances of increased risk of fall, injury, or foot drop.

According to aspects, an AFO device may assist dorsiflexion and provide feedback to a user based on integrated circuitry and sensors. By use of various combinations of compliant materials (e.g., flex joint, fiberglass, carbon fiber, and the like), an AFO device is able to adjust mechanical assistance provided to the user. AFO circuitry can monitor the user to provide critical feedback to assist locomotion. The AFO device may monitor a user's gait in real time using integrating sensors to detect the risk of falls, tripping, or foot drop. The integrated dynamic sensing is designed to distinguish between changes in gait (e.g., going uphill, downhill, upstairs, and downstairs) and actual risk of fall.

In accordance with aspects, an AFO device may be made of lightweight materials and an electronic feedback circuit. Example materials include, but are not limited to, a variety of composites, plastics, carbon fiber, fiberglass, KEVLAR®, plastic composites and/or other lightweight materials. Example electronic feedback circuit can include one or more biosensors, including but not limited to, goniometers, accelerometers, gyroscopes, force and pressure sensors, an electromyography sensors, and/or the like that are operatively connected to a microcontroller and/or feedback circuit which provides feedback using a speaker, buzzer, and/or vibrator. These components can be assembled into a working AFO device with integrated vibrotactile biofeedback.

AFO devices disclosed herein can simultaneously address stability, assisted walking, and feedback during locomotion. Further, AFO devices disclosed herein may be used as a therapeutic device in and outside of the clinical setting. AFO devices disclosed herein may provide critical feedback to the user, independent of a trained clinician. Further, an AFO device may be implemented, in part, by software configured for customization for user needs. In addition, an AFO device disclosed herein can be used to monitor gait, foot drop and other conditions that can lead to an user's fall or injury.

According to an aspect, an ankle-foot orthotic device may include one or more sensors configured to determine a gait phase of a user and to determine a movement of the user. Further, the device may include a controller configured to communicate feedback to the user based on the determined gait phase and the determined movement of the user.

### BRIEF DESCRIPTION OF THE DRAWINGS

The foregoing summary, as well as the following detailed description of various embodiments, is better understood when read in conjunction with the appended drawings. For the purposes of illustration, there is shown in the drawings exemplary embodiments; however, the presently disclosed subject matter is not limited to the specific methods and instrumentalities disclosed. In the drawings:

FIG. 1 is a side view of an AFO device with dynamic feedback and an integrated vibrotactile response in accordance with embodiments of the presently disclosed subject matter;

FIG. 2 is a flow chart of an example method for providing mobility feedback in accordance with embodiments of the present disclosure;

FIG. 3 is a side view of an AFO device being worn on a user's leg in accordance with embodiments of the presently disclosed subject matter;

FIG. 4 is a side view of an AFO device 100 being worn by the user while the user is walking downhill;

FIG. 5 illustrates a side view of an AFO device being worn on the user's leg while the user is walking uphill in accordance with embodiments of the presently disclosed subject matter;

FIG. 6 is a graph showing ankle angle measurement and feedback response thereto in accordance with embodiments of the presently disclosed subject matter; and

FIG. 7 illustrates corresponding graphs that show ankle angle and feedback intensity versus percent stride.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

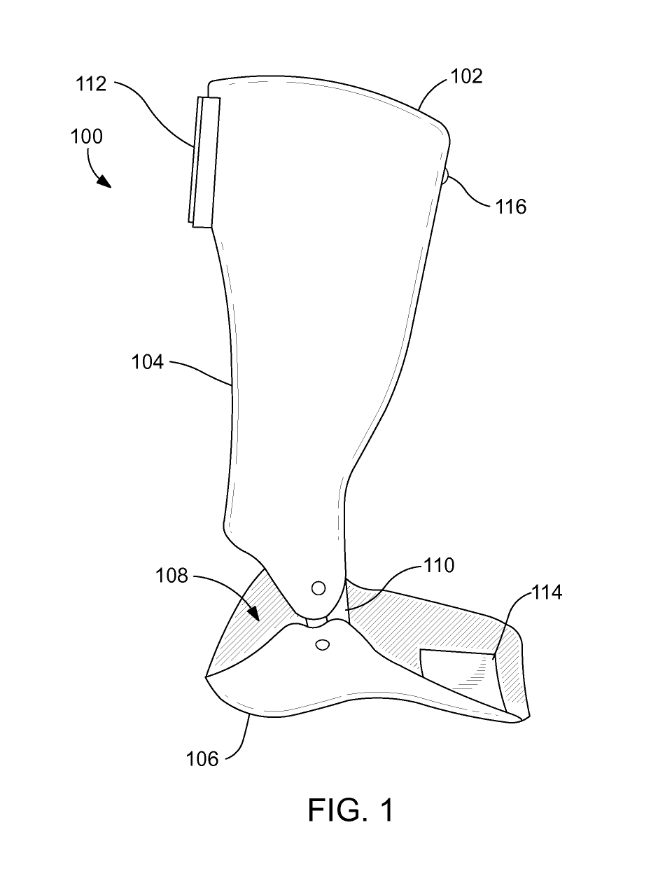

FIG. 1 illustrates a side view of an AFO device, generally designated 100, with dynamic feedback and an integrated vibrotactile response in accordance with embodiments of the presently disclosed subject matter. Referring to FIG. 1, the AFO device 100 may include a wearable mechanism 102 configured to fit to a leg and foot of a user. The mechanism 102 may include an upper portion 104 and a lower portion 106 that are connected together at a hinge portion, generally designated 108. The upper portion 104 may be sized and shaped to fit at least substantially around a lower leg of a user. In this way, the upper portion 104 can engage the lower leg (e.g., calf) of the user. The lower portion 106 may be sized and shaped to fit to a bottom surface of the user's foot or at least substantially surround the user's foot. In this way, the lower portion 106 can engage a foot of the user. The upper and lower portions 104 and 106 may be made of any suitable wearable material such as, but not limited to, carbon fiber, fiberglass, a variety of composites, plastics, a para-aramid synthetic material (e.g., KEVLAR®), plastic composites, other lightweight materials, or combinations thereof.

The hinge portion 108 may be made of any suitable flexible material and/or mechanism for allowing the user to move bend his or her ankle either entirely freely or with some restriction.

In accordance with embodiments, the AFO device 100 includes multiple sensors that are each configured to determine a movement and/or gait phase of a user. More specifically, the AFO device 100 includes a sensor 110 configured to determine a movement of the user. In the alternative, the sensor 110 may be referred to as a “biosensor.” The sensor 110 may be any suitable sensor capable of measuring an angle and/or angular velocity of the ankle of the user. Example sensors include, but are not limited to, a goniometer, potentiometer, strain gauge, accelerometer, gyroscope, and the like. The sensor 110 may be communicatively connected to a controller 112, and configured to communicate its measurements to the controller 112. The communication connection may be a wired or wireless connection. The measurement information may be communicated to the controller 112 via suitable signaling.

The AFO device 100 may include sensor 114 configured to determine a gait phase or mode of the user. The sensor 114 may be a kinematic or kinetic sensor configured to measure a force or pressure on a bottom surface of the foot of the user. In this example, the sensor 114 can be positioned beneath a front portion of the user when the AFO device 100 is being worn. A gait phase of a user may refer to a state of whether the user has a foot on the ground (stance) or not (swing). A gait phase of a user may refer to a state of whether the user is walking or running, or the user is standing still. The sensor 114 can determine that the user is in a state of standing when force applied to the sensor 114 is measured as being greater than a predetermined level (bodyweight) and not changing. In contrast, the sensor 114 can determine that the user is in a state of walking or running when force applied to the sensor 114 is measured as being greater than a predetermined level and changing dynamically with a given time signature. The sensor 114 may be communicatively connected to the controller 112, and configured to communicate its measurements to the controller 112. The communication connection may be a wired or wireless connection. The measurement information may be communicated to the controller 112 via suitable signaling.

The controller 112 may receive measurement signals from the sensors 110 and 114 for use in controlling a user interface 116 for providing feedback to the user. In this way, the controller 112 may communicate biofeedback for assisting in preventing foot drop. The controller 112 may be suitably configured with hardware, software, firmware, or combinations thereof for implementing functionality in accordance with the present subject matter. The controller 112 may receive the measurement information from sensors 110 and 114, and control the user interface 116 to communicate feedback to the user based on the determined gait phase and/or condition and determined movement of the user. For example, the user interface 112 may provide an auditory, visual, and/or vibratory feedback.

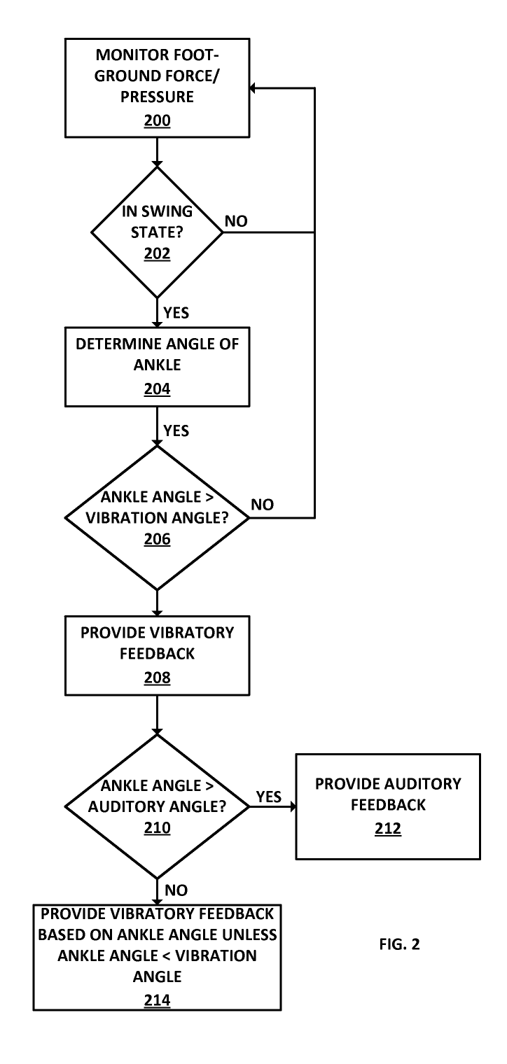

FIG. 2 illustrates a flow chart of an example method for providing mobility feedback in accordance with embodiments of the present disclosure. In this example, the method is described as being implemented by the AFO device 100 shown in FIG. 1; however, the method may alternatively be implemented by any suitable device or system. More particularly, the steps of this example method may be implemented by the controller 112 shown in FIG. 1.

Referring to FIG. 2, the method includes determining a force measurement by monitoring foot-ground force/pressure (step 200). For example, the controller 112 may receive a signal indicating a force/pressure measurement from the sensor 114. The method includes determining whether the user is in a swing state (step 202). The swing state can occur when the user is walking or running In this case, the leg wearing the AFO device 100 is moving without pressure being applied to the sensor 114 between the user's foot and the ground. This state is determined when the force measurement is zero or below a predetermined level. In response to determining that the force measurement is not zero and not below the predetermined level (i.e., the user is not in the swing state), the method may proceed to step 200. In contrast, in response to determining that the force measurement is zero or below the predetermined level (i.e., the user is in the swing state), the method may proceed to step 204.

At step 204, the method includes determining an angle of an ankle of the user. For example, the controller 112 may receive a signal indicating an angle of the ankle from the sensor 110. The method includes determining whether the ankle angle is greater than a predetermined vibration angle (step 206). In response to determining that the ankle angle is not greater than the predetermined vibration angle, the method may return to step 200. In contrast, in response to determining that the ankle angle is greater than the predetermined vibration angle, vibratory feedback may be provided (step 208). For example, the user interface 116 may be configured to provide a vibratory signal to the user, and the controller 112 may control the user interface 116 to provide the vibratory signal in response to determining that the ankle angle is greater than the predetermined vibration angle. Example vibration angles include angles greater than about 9 degrees, and/or less than about 13 degrees, or any other suitable angles.

The method of FIG. 2 includes determining whether the ankle angle is greater than a predetermined auditory angle (step 210). Example auditory angles include angles greater than about 13 degrees or any other suitable angles. In response to determining that the ankle angle is greater than the predetermined auditory angle, auditory feedback may be provided (step 212). For example, the user interface 116 may be configured to provide an auditory signal to the user, and the controller 112 may control the user interface 116 to provide the auditory signal in response to determining that the ankle angle is greater than the predetermined auditory angle. The auditory signal may be provided until the ankle angle becomes less than the predetermined auditory angle. In response to determining that the ankle angle is less than both the predetermined vibration and auditory angles, the method may return to step 200.

In response to determining that the ankle angle is not greater than the predetermined auditory angle, vibratory feedback may be provided based on the ankle angle unless the ankle angle is less than the predetermined vibration angle. For example, vibratory feedback may be provided at an increasing level as the ankle angle increases. The magnitude of the vibratory feedback may be controlled to correspond to a magnitude of the ankle angle. In this way, a user can realize, based on the vibratory feedback, that correction of movement is needed. The user can know that correction movement is more critical as the vibratory feedback level increases. Further, the user can know that movement correction is even more critical when auditory feedback is provided. In response to determining that the ankle angle is less than both the predetermined vibration and auditory angles, the method may return to step 200.

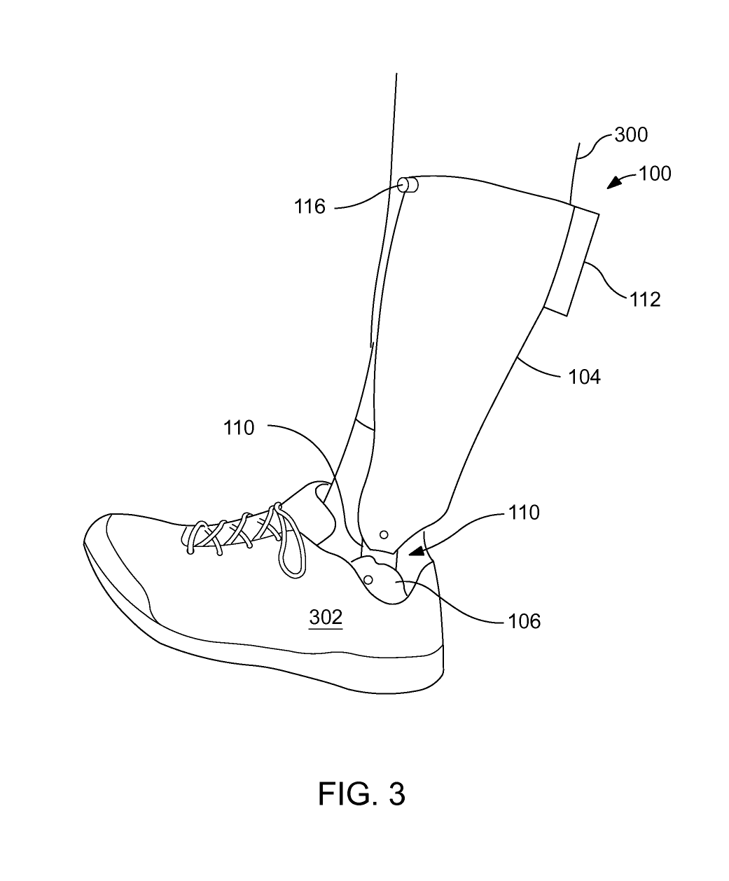

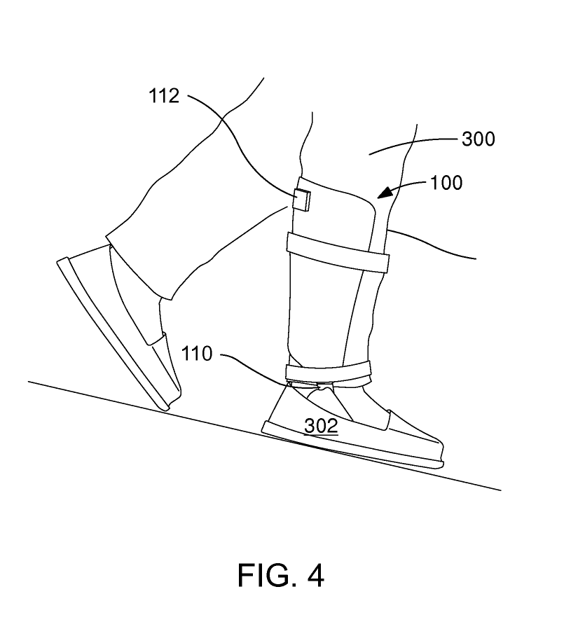

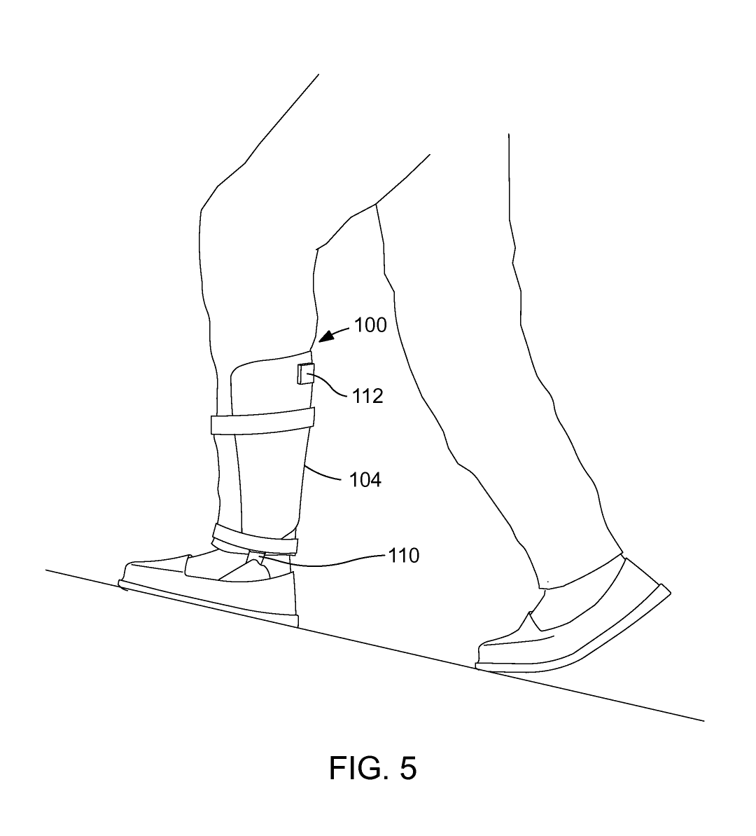

FIG. 3 illustrates a side view of an AFO device 100 being worn on a user's leg 300 in accordance with embodiments of the presently disclosed subject matter. Referring to FIG. 3, the lower portion 106 is mostly positioned within a shoe 302 being worn by the user. FIG. 4 illustrates a side view of an AFO device 100 being worn by the user while the user is walking downhill in accordance with embodiments of the presently disclosed subject matter. FIG. 5 illustrates a side view of an AFO device 100 being worn on the user's leg 300 while the user is walking uphill in accordance with embodiments of the presently disclosed subject matter.

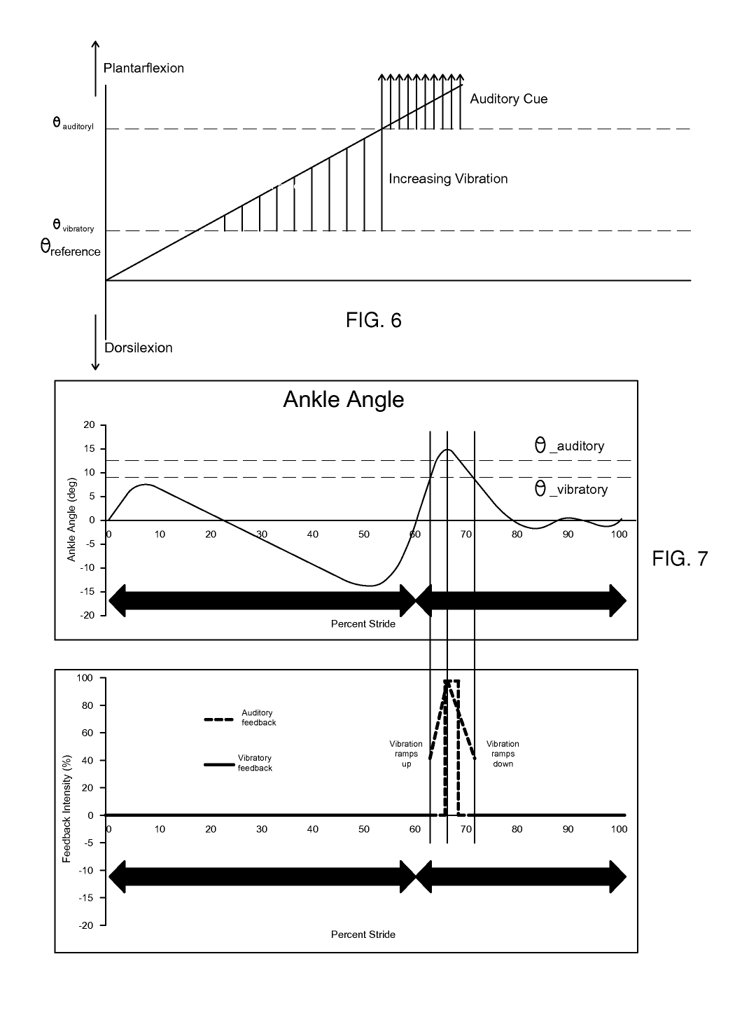

FIG. 6 illustrates a graph showing ankle angle measurement and feedback response thereto in accordance with embodiments of the presently disclosed subject matter. Referring to FIG. 6, an upward direction of the graph corresponds to increased plantarflexion, whereas a downward direction corresponds to increased dorsiflexion. When the ankle is positioned at an angle less than the vibratory angle θ_{vibratory}, there is no vibration or auditory cue or signal provided by the AFO device. At an angle between the vibratory angle θ_{vibratory} and the auditory angle θ_{auditory}, a vibration is provided and the vibration increases as the ankle angle increases. At an angle greater than the auditory angle θ_{auditory}, an auditory cue or signal is provided.

FIG. 7 illustrates corresponding graphs that show ankle angle and feedback intensity versus percent stride. Referring to FIG. 7, the figure shows an example ankle joint angle over a full walking gait cycle at a comfortable speed for a healthy individual from heel strike 0% to heel strike 100% of a single limb where 0 degrees represents the ankle angle during standing posture. The first 60% of the cycle is when the foot is in contact with the ground (i.e., stance phase), the second 40% is when the foot is not in contact with the ground (i.e., swing phase). The horizontal line labeled theta vibratory represents a threshold angle, above which, vibratory feedback proportional to the amount that ankle angle is above threshold would be given to the user (see the lower graph's bold trace for indication of timing and intensity of the resulting vibratory cue). The horizontal line labeled theta auditory represents a threshold angle, above which, auditory feedback of 100% intensity would be given to the user (see the lower graph's dashed trace for indication of timing and intensity of the resulting vibratory cue). Further, the graphs show the periods of stance (or standing) and swing phases, which are dependent upon the force or pressure detected by a sensor, such as the sensor 114 shown in FIG. 1. The upper graph shows an ankle angle (in degrees) over time. While in the swing phase, the ankle angle is used to determine the vibratory and auditory feedback as described herein. Vibration produced ramps up as the ankle angle increases, and the vibration produced ramps down as the ankle angle decreases. Further, at a high ankle angle above the auditory angle, auditory feedback is provided.

Although the examples provided herein relate to ankle angles, angle measurements of other joints may also be utilized. For example, a measure of the knee and/or hip, rather than the ankle, may be utilized in accordance with embodiments of the presently disclosed subject matter.

The various techniques described herein may be implemented with hardware or software or, where appropriate, with a combination of both. Thus, the methods and apparatus of the disclosed embodiments, or certain aspects or portions thereof, may take the form of program code (i.e., instructions) embodied in tangible media, such as floppy diskettes, CD-ROMs, hard drives, or any other machine-readable storage medium, wherein, when the program code is loaded into and executed by a machine, such as a computer, the machine becomes an apparatus for practicing the presently disclosed subject matter. In the case of program code execution on programmable computers, the computer will generally include a processor, a storage medium readable by the processor (including volatile and non-volatile memory and/or storage elements), at least one input device and at least one output device. One or more programs may be implemented in a high level procedural or object oriented programming language to communicate with a computer system. However, the program(s) can be implemented in assembly or machine language, if desired. In any case, the language may be a compiled or interpreted language, and combined with hardware implementations.

The described methods and apparatus may also be embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, a video recorder or the like, the machine becomes an apparatus for practicing the presently disclosed subject matter. When implemented on a general-purpose processor, the program code combines with the processor to provide a unique apparatus that operates to perform the processing of the presently disclosed subject matter.

Features from one embodiment or aspect may be combined with features from any other embodiment or aspect in any appropriate combination. For example, any individual or collective features of method aspects or embodiments may be applied to apparatus, system, product, or component aspects of embodiments and vice versa.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. An ankle-foot orthotic device comprising:
at least one sensor configured to determine a gait phase of a user and to determine a movement of the user; and
a controller configured to communicate feedback to the user based on the determined gait phase and the determined movement of the user.

2. The ankle-foot orthotic device of claim 1, wherein the at least one sensor comprises one of a goniometer, potentiometer, strain gauge, accelerometer, gyroscope, force sensor, and pressure sensor.

3. The ankle-foot orthotic device of claim 1, wherein the at least one sensor is configured to measure a force on a bottom surface of a foot of the user, and to communicate the measurement of the force or pressure to the controller, and
wherein the controller is configured to communicate feedback to the user based on the measurement of force or pressure.

4. The ankle-foot orthotic device of claim 1, wherein the at least one sensor is configured to determine an angle of a joint of the user, and to communicate the angle or angular velocity of the joint to the controller, and
wherein the controller is configured to communicate feedback to the user based on the angle or angular velocity of the joint.

5. The ankle-foot orthotic device of claim 4, wherein the angle of the joint is an angle of an ankle of the user.

6. The ankle-foot orthotic device of claim 1, wherein the at least one sensor comprises first and second sensors, the first sensor being configured to measure an angle of an ankle of the user, and the second sensor being configured to measure a force on a bottom surface of a foot of the user, and
wherein the controller is configured to:
determine whether the force/pressure measurement is less than a predetermined level;
in response to determining that the force/pressure measurement is less than the predetermined level, determine whether the angle measurement of the ankle meets a predetermined criterion; and
in response to determining that the angle measurement meets the predetermined criterion, communicate the feedback to the user.

7. The ankle-foot orthotic device of claim 6, wherein the ankle measurement includes one of a measurement of an angular velocity of the ankle

8. The ankle-foot orthotic device of claim 6, wherein the first sensor is configured to engage the ankle of the user.

9. The ankle-foot orthotic device of claim 6, wherein the second sensor is configured to engage the bottom surface of the foot of the user.

10. The ankle-foot orthotic device of claim 6, further comprising a mechanism configured to fit to a leg and foot of the user, and wherein the first and second sensors are attached to the mechanism.

11. The ankle-foot orthotic device of claim 1, wherein the at least one sensor comprises is configured to measure a force/pressure on a bottom surface of a foot of the user, and
wherein the controller is configured to:
determine whether the force/pressure measurement is greater than a predetermined level;
in response to determining that the force/pressure measurement is less than the predetermined level, preventing communication of feedback to the user.

12. The ankle-foot orthotic device of claim 1, wherein the controller is configured to vary the feedback communicated to the user based on one of the determined gait phase and the determined movement of the user.

13. A method for providing mobility feedback, the method comprising:
determining a gait phase of a user;
determining a movement of the user; and
communicating feedback to the user based on the determined gait phase and the determined movement of the user.

14. The method of claim 13, wherein determining a gait phase and determining a movement of the user comprises using at least one of a goniometer, potentiometer, accelerometer, strain gauge, gyroscope, force sensor, and pressure sensor.

15. The method of claim 13, wherein determining a gait phase of a user comprises measuring a force/pressure on a bottom surface of a foot of the user; and
wherein communicating feedback comprises communicating feedback to the user based on the measurement of force/pressure.

16. The method of claim 13, wherein determining a movement of the user comprises determining an angle of a joint of the user; and
wherein communicating feedback comprises communicating feedback to the user based on the angle of the joint.

17. The method of claim 16, wherein the angle of the joint is an angle of an ankle of the user.

18. The method of claim 13, further comprising providing first and second sensors,
wherein determining a movement comprises using the first sensor to measure an angle of an ankle of the user,
wherein determining a gait phase comprises using the second sensor to measure a force/pressure on a bottom surface of a foot of the user, and
wherein communicating feedback comprises:
determining whether the force/pressure measurement is less than a predetermined level;
in response to determining that the force/pressure measurement is less than the predetermined level, determining whether the angle measurement of the ankle meets a predetermined criterion; and
in response to determining that the angle measurement meets the predetermined criterion, communicating the feedback to the user.

19. The method of claim 13, further comprising providing a sensor,
wherein determining a gait phase comprises using the sensor to measure a force/pressure on a bottom surface of a foot of the user, and
wherein communicating feedback comprises:
determining whether the force/pressure measurement is greater than a predetermined level;
in response to determining that the force/pressure measurement is less than the predetermined level, preventing communication of feedback to the user.

20. The method of claim 13, further comprising varying the feedback communicated to the user based on one of the determined gait phase and the determined movement of the user.
